(19) United States
(12) Patent Application Publication
(10) Pub. No.: US 2024/0329025 A1
(43) Pub. Date: Oct. 3, 2024

(54) Electrochemical Analysis of Water with Multiplex Electrodes

(71) Applicant: Queen's University at Kingston, Kingston (CA)

(72) Inventors: Zhe She, Kingston (CA); Sarah Jane Odessa Payne, Kingston (CA); Yu Pei, Kingston (CA)

(21) Appl. No.: 18/619,477

(22) Filed: Mar. 28, 2024

Related U.S. Application Data
(60) Provisional application No. 63/456,317, filed on Mar. 31, 2023.

(51) Int. Cl. G01N 33/18; G01N 27/30

(52) U.S. Cl. CPC G01N 33/1813; G01N 27/301

(57) ABSTRACT

Methods and apparatus for analyzing water use a plurality of working electrodes and at least one counter electrode adapted to be in contact with the water, and a source of electrical potential difference that simultaneously applies to each of the plurality of working electrodes a different potential selected from at least one range of potentials. A multichannel device obtains chronoamperometry measurements from the plurality of working electrodes. The chronoamperometry measurements are used to generate a pattern of electrical charge on the plurality of electrodes, and the pattern of electrical charge is correlated with one or more known patterns to perform one or more of: identify one or more analytes in the water, perform speciation of analytes, and determine one or more concentration of the one or more analytes in the water.

### RELATED APPLICATION

This application claims the benefit of the filing date of Application No. 63/456,317, filed Mar. 31, 2023, the contents of which are incorporated herein by reference in their entirety.

### FIELD

The invention relates generally to the detection of impurities in fluids such as water. More specifically, the invention relates to electrochemical analysis methods and apparatus using multiplex electrodes for reliably identifying and accurately determining concentration of one or more analytes in fluids such as water.

### BACKGROUND

As an essential element naturally present in soil, water, and food, manganese (Mn) in trace amounts is important for sustaining physiological health. In drinking water distribution systems, Mn was previously considered a mere aesthetic issue that causes discoloration and an increase in the turbidity of water. Recently, epidemiological studies have confirmed the toxic effects of Mn on the central nervous system, causing neurological disorders and posing a risk to the neurodevelopment of children [1-3]. To address this emerging concern in drinking water, Health Canada created a new health-based maximum acceptable concentration (MAC) of Mn in drinking water as 0.12 mg/L (2.18 μM) to protect public health [4], and the World Health Organization (WHO) has also published a new provisional guideline of 80 μg/L [8]. Current laboratory methods, such as inductively coupled plasma mass spectrometry (ICP-MS) are sensitive and accurate methods to determine Mn²⁺ at trace levels [6]. However, in drinking water distribution systems, the sporadic release of Mn due to sudden hydraulic disturbance or change in chemical equilibrium or release from biofilm or release with biofilm is hard to predict and accurately monitor using current laboratory methods, because of the high cost for periodic monitoring, the transportation of samples to the laboratory, and the long waiting times for results. Manganese release events are also important to monitor as there can be co-releases of other accumulated metals such as arsenic, barium, chromium, lead, nickel, radium, uranium, vanadium [4], although this list is not exhaustive. Health Canada also recommends that iron also be monitored as manganese and iron often co-occur [4]. Manganese is important to monitor throughout the water treatment train and delivery system, i.e. from source to tap. A less expensive and more portable analytical method to detect Mn²⁺ is lacking.

Compared to laboratory methods, electroanalytical methods provide more rapid measurements and have the potential to be more compact than portable handheld devices. For Mn²⁺ detection, cathodic stripping voltammetry (CSV) is the electroanalytical method that has been demonstrated to be the most sensitive in detecting Mn²⁺ at trace levels [4, 8]. The sensitivity of CSV is due to the preconcentration step prior to the stripping step. Filipe et al. used carbon film electrodes fabricated from carbon resistors to determine the Mn²⁺ and achieved a limit of detection (LOD) of 4 nM [9]. Applying a microfabricated platinum (Pt) thin sensor with CSV can contribute to convenient on-site monitoring of Mn, as concluded by Kang et al [10]. However, the CSV method often faces hurdles of poor reliability due to the interference of other components in the drinking water matrix. To be specific, metal ions such as Fe²⁺, Pb²⁺, Cu²⁺, and Ni²⁺ cause interference with Mn²⁺ ions, and Fe²⁺ induces the most severe interference with the Mn²⁺ signal [9, 11-13]. Electrode modification with selective material is a common way to improve ion selectivity [14]. El-Desoky et al. proved that the presence of selected organic and inorganic species did not interfere with the CSV method coupled with oxine as a ligand for the determination of Mn²⁺ [12]. However, the low stability and reproducibility of the modified material renders it unsuitable for real-life applications.

### SUMMARY

According to one aspect of the invention there is provided a method for analyzing water, comprising: disposing a plurality of working electrodes and at least one counter electrode in the water; simultaneously applying to each of the plurality of working electrodes a potential selected from a range of potentials for a selected period of time; obtaining chronoamperometry measurements of the plurality of working electrodes during the selected period of time; using the chronoamperometry measurements to generate a pattern of electrical charge on the plurality of electrodes; and correlating the pattern of electrical charge with one or more known patterns to perform one or more of: identify one or more analytes in the water; perform speciation of analytes; and determine one or more concentration of the one or more analytes in the water.

One embodiment may comprise using a salt bridge between the water and a reference electrode.

One embodiment may comprise using a range of potentials from about −1.5 V to about 1.5 V.

One embodiment may comprise using a range of potentials from about 0.9 V to about 1.4 V.

In one embodiment increasing the selected period of time increases a detection sensitivity of analyzing the water.

In one embodiment the selected period of time is up to about 1 minute.

In one embodiment the selected period of time is up to about 5 minutes.

In one embodiment the selected period of time is greater than 5 minutes.

In one embodiment the analyte comprises one or more of manganese, copper, iron, lead, and arsenic.

In one embodiment the analyte comprises one or more of barium, chromium, nickel, radium, uranium, and vanadium.

One embodiment may comprise using two or more selected ranges of potentials; simultaneously applying to each of the plurality of working electrodes a potential selected from the two or more selected ranges of potentials for a selected period of time; obtaining chronoamperometry measurements of the plurality of working electrodes during the selected period of time; using the chronoamperometry measurements to generate a pattern of electrical charge on the plurality of electrodes corresponding to each of the two or more selected ranges of potentials; and correlating the patterns of electrical charge with known patterns to perform one or more of: identify two or more analytes in the water; perform speciation of analytes; and determine concentration of the two or more analytes in the water.

In various embodiments the water is drinking water, municipal water, well water, river water, lake water, or spring water.

In one embodiment the water is a water sample.

According to another aspect of the invention there is provided apparatus for carrying out methods and embodiments described herein.

In one embodiment the apparatus may comprise a plurality of working electrodes and at least one counter electrode adapted to be in contact with water being analyzed; a source of electrical potential difference that simultaneously applies to each of the plurality of working electrodes a different potential selected from at least one range of potentials; a multichannel device that obtains chronoamperometry measurements from the plurality of working electrodes; a processor that uses the chronoamperometry measurements to generate a pattern of electrical charge on the plurality of electrodes and correlates the pattern of electrical charge with one or more known patterns to perform one or more of: identify one or more analytes in the water, perform speciation of analytes, and determine one or more concentration of the one or more analytes in the water.

In various embodiments the apparatus may be adapted for analyzing individual water samples, or for implementation in a municipal water distribution system, a well, river, lake, or spring.

In one embodiment the apparatus may be implemented for in-line analysis of the water.

In one embodiment the apparatus may be implemented at least partially as a hand-held device.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1 is a flow chart of an experimental procedure of sample analysis according to one embodiment, comprising (a) electrochemical measurement of Mn²⁺ by multiplex chronoamperometry, (b) electrode acid cleaning using chronoamperometry, (c) rinsing electrodes with Milli-Q water and drying with nitrogen gas, (d) cleaned electrodes ready for the next electrochemical measurement.

FIGS. 2A-2E are diagrams showing a Mn²⁺ detection analysis and multichannel system experimental set-up according to embodiments, comprising (A) Mn2⁺ oxidation on Au, (B) MnO₂ reduction on Au, (C) a bottom view of an electrode holder with electrodes and salt bridge, (D) a front view of the experimental set-up, and (E) a system including capabilities for processing and partial or full automation.

FIGS. 3A and 3B are plots showing (A) reproducibility between 6 Au electrodes under the potential range from −0.1 to 0.6 V, and (B) CV of the same electrode in ferri/ferro cyanide solution on day 1 (freshly cleaned and polished), day 2, and day 6; no electrode polishing was done during the 6 days.

FIGS. 4A and 4B are plots showing charges of CA at Mn²⁺ concentrations of 0, 0.2, 0.4, 0.6, 0.8, 1.0 mM, wherein (A) shows linear calibration curves of Mn²⁺ detection under potential range from 0.9 to 1.4 V and (B) is a bar graph of the charges of CA at various Mn²⁺ concentrations.

FIGS. 5A and 5B are plots showing (A) CVs of 1 mM Mn²⁺, 1 mM Fe²⁺, and a mixture of 1 mM Mn²⁺ and 1 mM Fe²⁺, with a CV scan window of −0.3 to 1.2 V and a scan rate of 0.1 V/s, and (B) is a comparison of CA results of 1 mM Mn(II), 1 mM Fe(II), and the mixture of 1 mM Mn²⁺ and 1 mM Fe²⁺, wherein a range of CA potentials from 0.9 to 1.4 V were measured for each sample.

FIGS. 6A-6C are plots of the interference of various Fe²⁺ concentrations on 1 mM Mn²⁺ using concentrations of Fe²⁺ at 0, 0.2, 0.4, 0.6, 0.8, 1.0, 2.0, 5.0, 10, 20, 50, 100 mM spiked into 1 mM Mn²⁺; data are plotted at different ranges of Mn²⁺ concentrations, (A) from 0 to 100 mM, (B) from 0 to 1.0 mM, and (C) from 0.6 to 5 mM.

FIGS. 7A-7C show calibration curves of Fe²⁺ detection at different CA potentials, wherein the concentrations of Fe²⁺ at 0, 0.2, 0.4, 0.6, 0.8, 1.0, 2.0, 5, 10, 20, 50, 100 mM were analyzed; data are plotted at different ranges of Fe²⁺ concentrations, (A) from 0 to 100 mM, (B) from 0 to 1.0 mM, and (C) from 0.6 to 5 mM.

FIGS. 8A and 8B are plots showing pattern comparisons of Fe²⁺ samples with and without 1 mM Mn²⁺ wherein, for each pattern, the CA potentials from the left to right were 0.9, 1.0, 1.1, 1.2, 1.3, 1.4 V and Fe²⁺ concentrations added into 1 mM Mn²⁺ were (A) from 0.6 to 5 mM, and (B) from 10 to 100 mM, respectively.

FIGS. 9A and 9B are plots showing an example of using a multiplex pattern recognition method according to an embodiment described herein to determine the components of an unknown sample, wherein (A) shows the pattern of an unknown sample and (B) shows a bank of predetermined patterns of known samples.

FIG. 10 is a plot comparing patterns of samples of Mn²⁺ spiked in Milli-Q water (MQ) matrix and in drinking water (DW) matrix, wherein in each pattern from left to right the bars are charges obtained from CA at 0.9, 1.0, 1.1, 1.2, 1.3, 1.4 V and the samples from left to right were spiked with 0, 0.6, 1.0 mM Mn²⁺.

FIGS. 11A and 11B are plots of multiplex electrode charge patterns for three solutions with increasing concentrations of Mn²⁺ and charge patterns for three solutions with increasing concentrations of Cu²⁺, respectively.

### DETAILED DESCRIPTION OF EMBODIMENTS

One aspect of the invention relates to a method for identifying and/or quantifying one or more analytes in water using multiplex chronoamperometry (CA). According to embodiments, multiplex electrodes may be used to obtain CA measurements over a selected period of time, from which a charge pattern for the one or more analytes may be generated. Compared to conventional one-channel electrochemical techniques, a multiplex method as described herein generates a reliable charge pattern that is unique to the water sample components. According to embodiments, a generated charge pattern may be compared to known charge patterns to determine the identity and concentration of the one or more analytes. In some embodiments, an electrochemical database including charge patterns of known analytes may be used, and a generated charge pattern may be compared to charge patterns in the database to determine the identity and concentration of the one or more analytes.

In some embodiments, comparisons of generated patterns with known patterns for identification of analytes in samples and/or the concentrations of analytes may be performed using pattern recognition software running on a processor such as a computer. The processor may include a non-volatile memory device storing computer code containing instructions that direct the processor perform processing steps such as, but not limited to, accessing raw or preprocessed measurement data, generating charge patterns, comparing charge patterns with known charge patterns for various analytes and their concentrations, and outputting a result such as the identity of one or more analytes in the sample, the concentration of one or more analytes in the sample, etc. In some embodiments the memory device may store a database or library of known patterns and the processor may access the stored patterns to facilitate the comparisons.

FIG. 2E is a diagram showing an implementation of multichannel potentiostat system for at least partially automating sample analysis, according to one embodiment. Referring to FIG. 2E, a testing apparatus 250 comprises a plurality of working electrodes 252, a counter electrode 254, and optional salt bridge 256 and reference electrode 258. In some embodiments the testing apparatus 250 may include a sample container for containing the sample (e.g., water) being analyzed. In other embodiments the testing apparatus may be configured so that it can be disposed in a water source to be analyzed in a way that avoids the need to obtain a discrete water sample. For example, the testing apparatus may be configured to attach directly to a water conduit of a water distribution system, or it may be configured to be disposed in a lake, river, reservoir, well, etc., either as a permanent or semi-permanent “in-line” system for continuous scheduled water testing, or at least partially automated water testing, or for single discrete measurements. In such embodiments the testing apparatus 250 may be easily changed/replaced/serviced as needed. In some embodiments the testing apparatus 250 may be reusable after cleaning/reconditioning the electrodes using, e.g., a procedure as described herein. In other embodiments the testing apparatus 250 may be disposable.

The electrodes are connected to a device 260 that performs electrochemical multiplex analysis, such as a chronoamperometry device that applies potentials to the plurality of electrodes and measures the charge on the electrodes, such as electrochemical analyzer. Measurements obtained by the device 260 may be output directly to a processor (i.e., a computer) 280, or, depending on the type of output from the device 260, preprocessing 270 may optionally be performed on the output measurements, such as, for example, analog-to-digital conversion, prior to input into the processor 280. In some embodiments any preprocessing required may be carried out by the processor 280. The processor may optionally send commands, instructions, etc. 285 to the device 260. The processor may include non-transitory computer readable storage media having stored thereon instructions that when executed by the processor, cause the processor to execute processing steps which may include receiving the measurement data, analyzing the data, identifying one or more analytes from the data, determining a concentration of one or more analytes in the sample, and outputting result of such analyses to an output device 290 such as a display screen. The processor 280 and output device 290 may support a graphical user interface (GUI), and the output device may comprise a touch screen to allow user input and control. Alternatively, and/or additionally, an input device 295 such as a keyboard, mouse, etc. may facilitate user input and control. User input and control may comprise the user entering commands and selecting menu options to set measuring parameters, set scheduling of automated measurements, and the like. Embodiments of a system incorporating components, e.g., as shown in FIG. 2E, may be implemented in various forms such as compact hand-held and bench top systems, or as large scale distributed systems wherein, for example, components such as the processor and input/output devices may be located some distance from the measuring device 260, which may include wireless connections between some components.

Although embodiments are described herein primarily with respect to detecting and quantifying Mn²⁺, it will be appreciated that the invention is not limited thereto. Other analytes, including those which may be referred to as contaminants, may also be detected and quantified according to methods provided herein. Examples include but are not limited to Cu²⁺, Fe²⁺, Fe³⁺, Pb²⁺, As³⁺, etc. Further, manganese release events may co-release other accumulated metals such as, for example, barium, chromium, nickel, radium, uranium, and vanadium. Accordingly, embodiments may include detecting and quantifying one or more of barium, chromium, nickel, radium, uranium, and vanadium separately and/or in addition to one or more of manganese, copper, iron, lead, and arsenic.

Embodiments are described herein primarily with respect to detecting one or more analytes in water. In various embodiments and implementations, the water may be drinking water (i.e., potable water), water obtained from a distribution system referred to herein as municipal water, well water, water from a source such as a river, lake, pond, spring, etc. Embodiments may be adapted for use in other liquids such as organic solvents and in fluids such as gases.

In general, using multiplexed working electrodes, multiple potentials are applied to the electrodes to conduct CA analysis. The potential applied to each electrode may be selected from a range of potentials, for example, from about −1.5 V to about +1.5 V, although other ranges may be used. To improve sensitivity of the measurements, a narrower range may be selected as more appropriate for an analyte of interest, for example, about 0.9 V to about 1.4 V for an analyte such as Mn²⁺. Also to improve sensitivity, the number of electrodes may be increased, such that smaller voltage differences between electrodes may be used. For example, in the case of 0.9 V to about 1.4 V, six electrodes may be used wherein the potentials applied to the six electrodes are 0.9, 1.0, 1.1, 1.2, 1.3, and 1.4 V, or 12 electrodes may be used wherein the potentials applied to the 12 electrodes are 0.90, 0.95, 1.0, 1.05, . . . , 1.30, 1.35, and 1.40 V. In general, fewer than three electrodes would impose a significant restriction on the range of potentials that could be used, whereas a large number of electrodes (e.g., 20-50) allows either a wide range of potential or a fine resolution within a selected range of potential. It is noted that microfabrication techniques may be employed to produce chips or other structures with large numbers of electrodes. Thus, the maximum number of electrodes may be limited only by what is practical to implement for a given application. In some embodiments electrodes may be grouped according to two or more different selected ranges of potentials, to facilitate detection of two or more different analytes within a sample.

In some embodiments, there may be more electrodes than the number of channels available on a CA instrument (such as a potentiostat), or in certain implementations it might not be practical to have a CA instrument with a corresponding large number of channels. In such embodiments CA measurements may be carried out by applying the potentials to and obtaining measurements from subsets of electrodes sequentially. The measurements may be carried out in rapid succession wherein a pattern is generated with substantially the same result, so that the measurements may be considered effectively or substantially simultaneous. Accordingly, embodiments may include applying potentials and obtaining measurements substantially simultaneously.

As used herein, the term “multiplex” refers to applying potentials to more than one electrode and obtaining measurements from the more than one electrode simultaneously and/or substantially simultaneously.

The potentials may be applied and CA measurements on the electrodes carried out for a selected period of time. According to embodiments, the selected period of time can be short, e.g., 1 ms or less, or long, e.g., up to 1 s, or 1 min, or 5 min, or longer. A CA measuring instrument may acquire measurements/data points during the selected time. Overall sensitivity of embodiments may be enhanced with longer selected periods of time, although it is recognized herein that a longer period of time may reduce sample processing. In comparison with cyclic voltammetry (CV) that scans within a potential window, the use of CA as described according to embodiments provides significantly greater detail in the characteristics of each potential by accumulating the signal over the selected period of time.

Methods described herein may also reduce metal interference without surface modification. For example, data generated from multiple CA analyses may be used to generate a unique pattern for an analyte such as Mn²⁺ at different concentrations with the presence of Fe²⁺. Fe²⁺ is a common component in drinking water. In addition to the naturally occurring Mn²⁺ and Fe²⁺ in surface water, water distribution system pipes can accumulate metals such as Mn and Fe in treated drinking water when it passes through, creating a reservoir that can later be released. Additionally, Fe can be released through corrosion of cast iron pipes, increasing the concentration of Fe in drinking water. The presence of Fe²⁺ interferes with Mn²⁺ voltammetric detection, even at an interference level lower than one. The interference is due to Fe²⁺ and Mn²⁺ having similar electrochemical properties such as their redox activity at the given potential window. However, Fe²⁺ oxidizes more rapidly than Mn²⁺, which leads to Fe²⁺ receiving more electrons than Mn²⁺ during oxidation. The preference of electrons towards Fe²⁺ can be explained by Fe (2957 KJ/mol) having a lower third ionization energy than Mn (3248 KJ/mol). Therefore, the interference of Fe²⁺ may be used to determine characteristic patterns and distinguish between Fe²⁺ and Mn²⁺ signals. This is described in detail in the below Example. Other interfering components may similarly be characterized and detected in samples using the principles and methods described herein.

Further aspects and features of the invention will be apparent from the below examples which describe certain embodiments. It will be understood that the scope of the invention is not limited by the Examples.

### Example 1

Embodiments of an implementation and its use in analyzing a water sample will now be described.

### Chemical and Reagents

Manganese sulfate monohydrate (≥99%), iron (II) sulfate heptahydrate (≥99%), potassium hexacyanoferrate (II) trihydrate (98.5-102.0%) and sodium perchlorate monohydrate (≥98%) were obtained from Sigma-Aldrich (Oakville, ON). Potassium ferricyanide (99%), sulfuric acid (trace metal grade, 93-98%), potassium hydroxide (trace metal basis, 99.98%), agar (molecular genetics), nitric acid (99.999%) and potassium nitrate (99%) were obtained from Thermo Fisher Scientific (Ottawa, ON). Prior to use, Milli-Q water with a resistivity of 18.2 MΩ·cm was passed through a 0.22 μm filter.

### Sample Preparation

A ferri/ferrocyanide analyte solution was prepared by dissolving potassium hexacyanoferrate (II) trihydrate (5 mM), potassium ferricyanide (5 mM), and sodium perchlorate monohydrate (1 M) in 40 mL Milli-Q water. Except for the ferri/ferrocyanide analyte solution, all the other samples were prepared using Milli-Q water containing 0.1 M potassium nitrate as a supporting electrolyte. The samples were prepared in 10 mL or 50 ml centrifuge tubes, using the serial dilution method. A drinking water sample was collected from a municipal water tap in Kingston, Ontario, Canada. The sample was collected after flushing the tap for 5 minutes. Before testing, the drinking water sample was set in an open container at room temperature overnight to remove chlorine and the acidity was adjusted to pH 6 using 20% nitric acid.

### Electrode Preparation

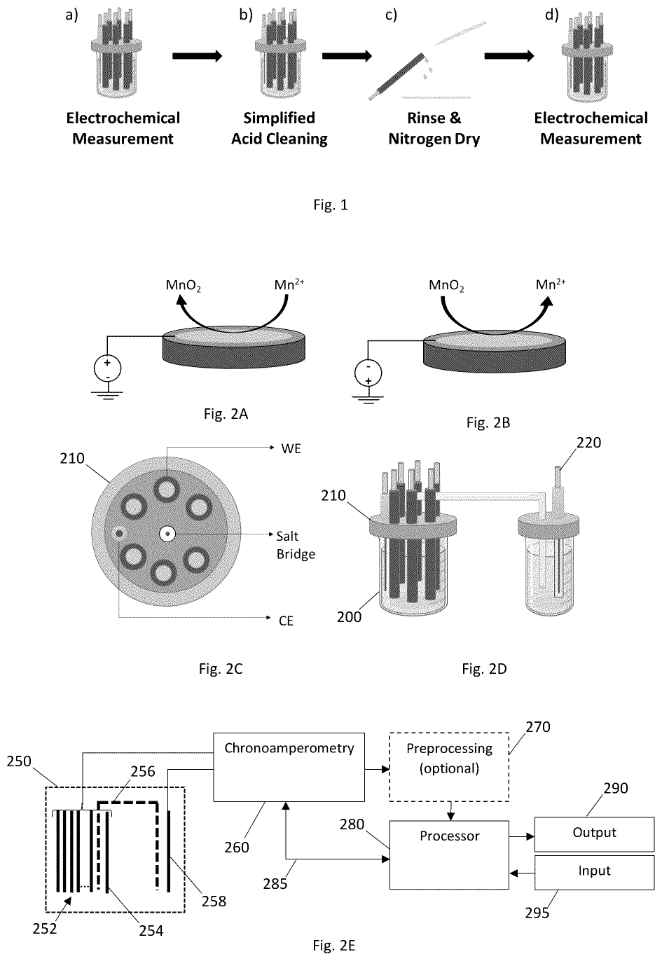

The electrode cleaning procedure shown in the embodiment of FIG. 1 was used. Firstly, each electrode was polished for 3 minutes with 0.3 and 0.05 μm alumina powder and polishing pads purchased from Allied High Tech Products, Inc. (Rancho Dominguez, CA, U.S.A.). Next, the electrodes were sonicated for 10 minutes using an ultrasonic bath (Fisher Scientific, Mexico). Then the electrodes were inserted into acid (0.5 M H₂SO₄) and base (0.5 M KOH) solutions and connected to the CH instrument program for cleaning purposes (10 minutes in acid and 13 minutes in base solution). The final step of cleaning was determining the cleanliness by comparing the cyclic voltammetry of 6 electrodes in an analyte solution containing 5 mM ferricyanide/ferrocyanide, and 1 M NaClO₄.

### Multiplex Set-Up and Measurement

FIGS. 2A and 2B are diagrams showing Mn²⁺ oxidation on Au and MnO₂ reduction on Au, respectively. FIGS. 2C and 2D are diagrams of an experimental set-up of a multichannel potentiostat system, according to an embodiment. A 20 ml beaker was used as the solution container 200 and a 3-D printed electrode holder 210 composed of polylactic acid (PLA Silver) was produced by a Josef Prusa 3-D printer. The 3-D printed electrode holder was designed with smaller holes for the salt bridge and the counter electrode (CE), and six larger holes around the salt bridge for inserting the working electrodes (WE). The gold working electrodes (CHI101), the platinum wire counter electrode (CHI115), and the Ag/AgCl in 3 M KCl reference electrode were purchased from CH Instrument (CH Instrument, Texas). During the measurements, the counter electrode and six working electrodes were immersed into the solution at the same depth and the salt bridge connected the analyte container to the external reference electrode 220. The electrochemical multiplex analysis was located in a faraday cage and was measured by a CHI1030C Electrochemical Analyzer (CH Instrument, Texas). Potentials of 0.9, 1.0, 1.1, 1.2, 1.3, 1.4 V were applied to the six electrodes simultaneously for 3 minutes by CA, respectively.

### Simplified Electrode Cleaning

For simplified electrode cleaning, CA scans were run on electrodes in 0.1 M H₂SO₄ solution for 5 minutes with an initial potential of −1.0 V, then the electrodes were rinsed with Milli-Q water and air-dried with nitrogen gas. This cleaning step was required during every measurement.

### Electrode Reproducibility and Stability Test

Reproducibility of the Au electrodes was tested by running CV measurements of the six electrodes in an analyte solution within the potential range from −0.4 to 0.7 V using a scan rate of 0.1 V/s. A simplified cleaning step was required during each measurement and this two-step process was repeated seven times. The oxidation peak currents for each CV were determined and underwent statistical tests to evaluate the stability of the Au electrodes over a series of days, CVs of the six electrodes in analyte solution were conducted on Day 1, Day 2, and Day 6, with only the simplified cleaning procedure being used before every measurement.

### CV Measurement

A potentiostat (CHI 420C, CH Instrument, Texas) was used to obtain the CVs of Mn²⁺ and Fe²⁺ sample solutions. Samples containing different concentrations of Mn²⁺ and Fe²⁺ were diluted with 1.0 M KNO₃, which acted as a supporting electrolyte. The potential range of the voltammogram was −0.4 to 1.2 V with a scan rate of 0.1 V/s. All 0.2 mm diameter gold disc-rod working electrodes, a platinum wire counter electrode and a Ag/AgCl in 3 M KCl reference electrode (CH Instrument, Texas) were used for the CV measurements. The electrochemical system was set up by the working and counter electrode in the same beaker with the sample solution, while a salt bridge connected them to a reference electrode.

### Statistical Analysis

The data analysis was processed by IBM SPSS software (Armonk, NY). The results showed significance while using the 95% confidence interval.

### Limit of Detection (LOD) Determination

The blank solution used to determine the LOD was 0.1 M KNO₃ in Milli-Q water. CA measurement was carried out in the blank solution and simplified acid cleaning (0.1M H₂SO₄) was done between each measurement. The measurement was repeated seven times to acquire the data. The LOD value was determined by Equation (1):

$$LOD = 3 σ / slope \qquad ( 1 )$$

Where σ is the standard deviation of the seven replicated measurements and the slope is the slope of the calibration curve.

### Results

### Reproducibility Test of Electrodes and Set-Up Test

FIG. 3A shows the well-overlapped CVs of the six electrodes in 0.5 M ferri/ferrocyanide solution and 1 M NaClO₄ under the potential range from −0.2 to 0.7 V, which indicates high reproducibility among the Au electrodes. All six electrodes were immersed in the same analyte solution in the same electrode cell, as shown in FIG. 2D. The oxidation peak current of CV was determined as the highest current of the oxidation peak at 0.2976 V. Three peak currents were measured for each electrode, and a one-way ANOVA test was conducted to determine if the difference between the peak currents of each electrode is significant. The significance obtained from the statistical test was 0.673, which showed the differences in the peak currents between each electrode were not significant (a=0.05).

The stability of the Au electrodes was evaluated by storing six freshly cleaned and polished electrodes under ambient conditions for one week. The cleanliness of the Au surface was determined by observing the change in redox peaks in 0.5 M ferri/ferrocyanide and 1 M NaClO₄ solution by CV. The CVs of the six Au electrodes for the first, second, and sixth days were measured, respectively. The results in FIG. 3B indicate that the Au electrodes maintain stability for at least one week, without the need for additional electrode polishing for cleaning and regenerating the electrode surface.

### Mn²⁺ Determination by CA

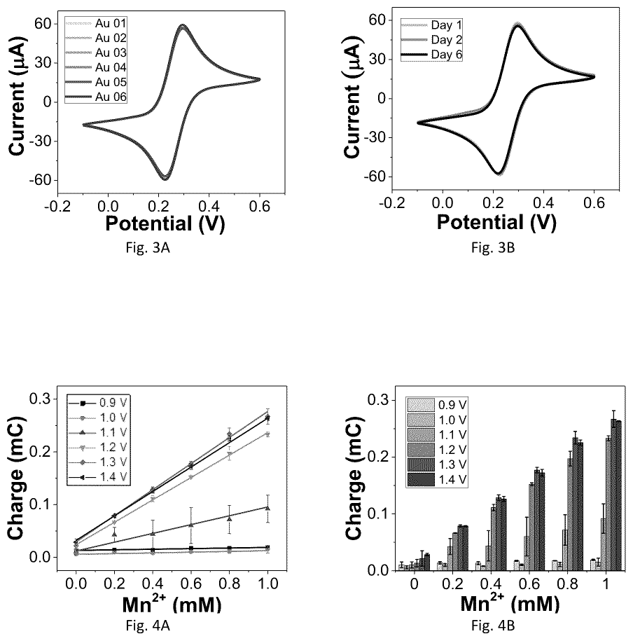

The Mn²⁺ solutions with various concentrations were analyzed at different potentials by CA. For each Mn²⁺ concentration, the only variable for the CA measurements is their potential, ranging from 0.9 V to 1.4 V. The currents during the entire CA scan were integrated as the charge for making the calibration curve. The charges generated from CA at different Mn²⁺ concentrations show a linear or near-linear trend for potentials from 0.9 V to 1.4 V, as shown in FIG. 4A. The linear equations and R² values of the linear fits are shown in Table 1.

TABLE 1
Summary of Calibration Curves Under Different Potentials.

| Potential (V) | Calibration Curve* | | R² | LOD (μM) |
|---|---|---|---|---|
| 0.9 | y = 0.00567x + 0.0131 | (2) | 0.662 | 469 |
| 1.0 | y = 0.00698x + 0.00554 | (3) | 0.481 | 814 |
| 1.1 | y = 0.0835x + 0.0118 | (4) | 0.923 | 53.1 |
| 1.2 | y = 0.212x + 0.0239 | (5) | 0.998 | 25.6 |
| 1.3 | y = 0.247x + 0.0292 | (6) | 0.998 | 27.5 |
| 1.4 | y = 0.232x + 0.0320 | (7) | 0.999 | 32.4 |

*In Equation (2) to (7), the x values represent the concentration in moles per liter of Mn²⁺, and the y values represent the charge in millicoulombs of CA.

The Mn²⁺ calibration curve of 1.2, 1.3 and 1.4 V is linear, with the R² values over 0.99. This reveals an excellent linear fit of the data points, which indicates that the oxidation of Mn²⁺ occurs at those potentials and the oxidation is linearly correlated to the concentration of Mn²⁺ in the range from 0 to 1.0 mM. At 1.1 V potential, the trend in increased linearity was also observed for the Mn²⁺ solution; however, the error bars of 1.1 V are larger than the errors of potentials above 1.1 V. Larger error bars indicate the generation of less stable oxidation products, which is consistent with the oxidation mechanism of Mn²⁺. The oxidation of Mn²⁺ follows a three-step mechanism. The Mn²⁺ ions first get oxidized to Mn³⁺ ions, which is not stable in aqueous systems [4]. The Mn³⁺ ions convert rapidly to MnOOH as a precipitate. When a higher potential is applied, MnOOH is further oxidized to solid state MnO₂. When 1.2 V to 1.4 V potentials are applied to the electrode, Mn²⁺ is oxidized to a stable MnO₂ film and deposited on the electrode surface. The formation of Mn³⁺ is less stable than MnO₂, which results in larger error bars. When applying potentials of 0.9 and 1.0 V, the linear fittings have low slopes that indicate the charge transfer from Mn²⁺ oxidation is small at potentials lower than 1.0 V. According to Table 1, the LOD decreases when increasing the CA potentials. This indicates that the oxidation process is more complete under higher potentials, whereas more Mn²⁺ signals can be detected. The best LOD obtained from the range of potentials applied was 25.6 μM when the potential was 1.2 V. The LOD of the measurements may be further improved by applying longer CA deposition time or using a different electrode material.

The data from the calibration curves of FIG. 4A is replotted as bar plots in FIG. 4B. Instead of showing changes in charge at different Mn²⁺ concentrations under certain potentials, FIG. 4B is plotted to show the difference of charges at different potentials under a certain Mn²⁺ concentration. Using the charges at multiple CA potentials, a unique pattern is generated for each Mn²⁺ concentration. The general trend of the patterns is that the higher the concentration of Mn²⁺, the taller the bars. This trend is consistent with the linear or near-linear calibration curves of different CA potentials, as shown in FIG. 4A. Under a certain Mn²⁺ concentration, the potential of 1.3 V shows the strongest signal, as the tallest bar in the pattern. This is also consistent with FIG. 4A, where the calibration curve of 1.3 V is the tallest among the other calibration curves.

### Exploration of Iron Interference

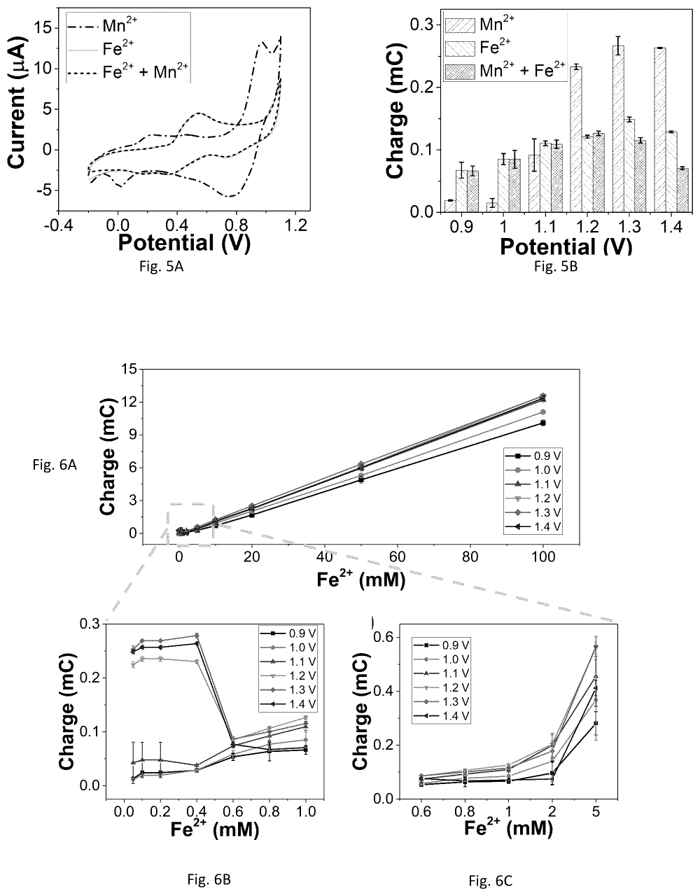

The CVs of Mn²⁺ (dash-dot line) and Fe²⁺ (solid line) were measured as shown in FIG. 5A. The peak currents of 1 mM Mn²⁺ oxidation is observed around 0.2205 V and 0.9760 V, respectively. Each peak corresponds to the oxidation mechanism of Mn²⁺. The oxidation peak of Fe²⁺ is observed around 0.5415 V, which represents the oxidation from Fe²⁺ to Fe³⁺. The CV of a mixed solution of 1 mM Mn²⁺ and 1 mM Fe²⁺ (dashed line) was obtained to explore the interference of Fe²⁺ to Mn²⁺. Interestingly, as shown in FIG. 5A, the CV of Mn²⁺ (1 mM) and Fe²⁺ (1 mM) mixture solution completely overlaps with pure Fe²⁺ (1 mM) solution, and the oxidation peaks of Mn cannot be observed. This indicates that when 1 mM Fe²⁺ is present in the sample solution, 1 mM Mn²⁺ cannot be detected by CV. This confirms that Fe²⁺ interferes with Mn²⁺ voltammetric detection. The interference of Fe²⁺ with Mn²⁺ is further explored by CA measurements.

The CAs of 1 mM pure Mn²⁺, 1 mM pure Fe²⁺ and the same Mn²⁺ and Fe²⁺ mixture solution at different potentials were measured, as shown in FIG. 5B. The CA results provide a more detailed illustration of Fe interference at each potential. Comparing the charges of pure Mn²⁺, the charges of the mixture solution are larger at the potentials from 0.9 to 1.1 V and smaller at the potentials from 1.2 to 1.4 V. The differences in charges indicate the presence of Fe²⁺ interferences in the CA analysis of Mn²⁺ in the potential range of 0.9 V to 1.4 V. The same CA analysis at different potentials was also conducted for pure 1 mM Fe²⁺ solution. In FIG. 5A, the CVs of pure 1 mM Fe²⁺ and the mixture of 1 mM Fe²⁺ and 1 mM Mn²⁺ are identical; however, the CAs are not entirely the same (FIG. 5B). The charges from 0.9 to 1.2 V are similar between pure Fe²⁺ and the mixture, while the charges of 1.3 V and 1.4 V are different. Due to the difference of the overall pattern, this indicates that the pattern recognition method may be used to determine the concentration of Mn²⁺, even with the presence of Fe²⁺.

CA Analysis of Mn²⁺ in the Presence of Fe²⁺

In order to understand the interference of Fe²⁺ on Mn²⁺ CA analysis, different concentrations (0, 0.2, 0.4, 0.6, 0.8, 1.0, 2.0, 5.0, 10, 20, 50, 100 mM) of Fe²⁺ were spiked into 1 mM Mn²⁺ solution for the analysis. As shown in FIG. 6A, from 0 to 100 mM Fe²⁺ in 1 mM Mn²⁺ solution, the increase of charges measured from CAs appear to be linear when 0.9 to 1.4 V potentials were applied. However, the enlarged plots of lower concentrations shown in FIGS. 6B and 6C demonstrate the decrease of charge when a large amount of Fe²⁺ is introduced into the system. When concentrations ranging from 0 to 0.4 mM Fe²⁺ were added to 1 mM Mn²⁺, the charge transfer at all six CA potentials stays the same. The interference is small when the stoichiometry between Fe²⁺ and Mn²⁺ is smaller than 0.4. As also shown in FIG. 6B, when 0.6 mM Fe²⁺ was added into the 1 mM Mn²⁺ solution, the charges from 1.2 to 1.4 V CA dropped conspicuously. At the same time, at potentials from 0.9 to 1.1 V, the charges gradually increase with the increase of Fe²⁺ concentration. As discussed previously, the stability of the Mn²⁺ oxidation reaction only occurred when potentials at 1.2 V and above were applied. The drops of CA signals in FIG. 6B indicate a strong interference of Fe²⁺ with Mn²⁺ oxidation above the stoichiometry of 0.6. Overall, the stoichiometry of 0.5 between Fe²⁺ and Mn²⁺ appears to be the turning point of Mn²⁺ oxidation. When the concentration of Fe²⁺ is higher than 0.6 mM, charges obtained from all CA potentials steadily increase with a curve, as shown in FIG. 6C. A question arises if only Fe²⁺ is contributing to the increase of the charge. To explore the correlation between the charges of the mixture solution and the charges of pure Fe²⁺ solution, the same CA analysis was also carried out for pure Fe²⁺ solution.

### Comparison Between Mixture and Pure Fe²⁺ Solution

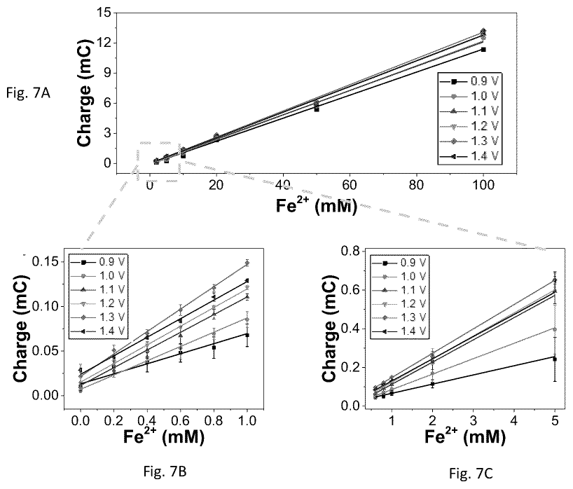

To determine if there is a difference between the charges of pure Fe²⁺ and the mixture containing Mn²⁺, a series of Fe²⁺ solutions were analyzed using the same CA method. As shown in FIG. 7A, a similar linear correlation is observed for pure Fe²⁺ solutions. The difference is the enlarged figures of low Fe²⁺ concentrations, shown in FIGS. 7B and 7C, also demonstrate linear correlations. The sudden >0.1 mC decreases in charge observed in FIG. 6B and the non-linear increases of FIG. 6C were not observed in FIGS. 7B and 7C. All the calibration curves have a strong linear correlation with R² values above 0.999 and all have similar slope values around 0.12 (unit of C/M). When analyzing Mn²⁺ solutions by the same method, only calibration curves with CA potentials between 1.2 and 1.4 V had R² values above 0.999 and overlapping calibration curves. This was due to the fact the stable oxidation of the Mn²⁺ reaction happens when the potential is higher than 1.2 V. For Fe²⁺, a strong linear correlation happens at lower potentials even at 0.9 V, which indicates the oxidation of Fe²⁺ requires a lower oxidation potential than Mn²⁺. The low oxidation potential of Fe²⁺ is also supported by the CV of Fe²⁺ as shown in FIG. 5A, where the oxidation peak of Fe²⁺ is at 0.5415 V and the strongest oxidation peak of Mn²⁺ is at 0.9760 V. The differences between pure Fe²⁺ and the mixture solution at low Fe²⁺ concentrations support that the abnormal drop of the calibration curves in FIG. 6B was due to the interaction between Fe²⁺ and Mn²⁺. The results also prove that at stoichiometry below 0.4, Mn²⁺ is more dominant in terms of determining the charge transfer of CA analysis. Between the stoichiometry of 0.6 and 5, the non-linear correlation indicates that the presence of Mn²⁺ also has an impact on the Fe²⁺ oxidation. The correlation of charge is not exclusively determined by the concentration of Fe²⁺.

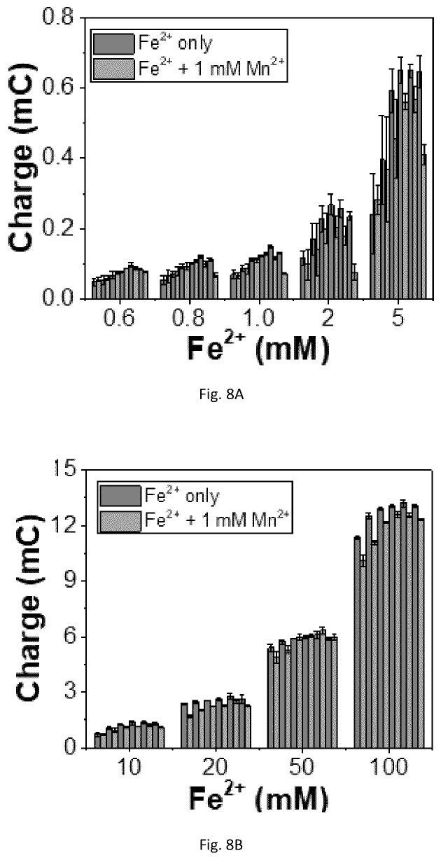

The data from the calibration curves were replotted as bar plot patterns at Fe²⁺ concentrations after the turning point at 0.6 mM, as shown in FIGS. 8A and 8B. The patterns created by the individual bar plots from pure Fe²⁺ and the mixture solution containing Mn²⁺ are compared. When Fe²⁺ concentrations are between 0.6 and 1.0 mM, the difference of the bar plots from 0.9 to 1.2 V between pure Fe²⁺ and mixture solution is not significant (α=0.05). However, there is a significant difference between the two types of solutions at 1.3 and 1.4 V. The charges of the mixture solution at 1.3 and 1.4 V are lower than the charge of pure Fe²⁺, which is due to the interaction between Mn²⁺ and Fe²⁺, as discussed previously. When increasing the Fe²⁺ concentration with a fixed Mn²⁺ concentration of 1 mM, the difference is more obvious and gradually causing a significant difference at lower CA potentials as well (shown in FIG. 8B).

Because of the differences in the patterns, comparing an unknown Mn²⁺ sample in the presence of Fe²⁺ with the predetermined database of patterns solves the problem of being unable to determine Mn²⁺ in presence of Fe²⁺ by current voltammetric methods, such as CV and CSV. A multiplex method according to embodiments described herein uses CA to accumulate and enhance current signals obtained at different potentials on multiple electrodes to generate a unique pattern for a particular sample and its matrices. Based on this principle, a database library of these patterns may be developed and used to identify various analytes and sample properties. This approach has not been found to be documented in literature.

### Pattern Recognition Method of Mn²⁺ Detection

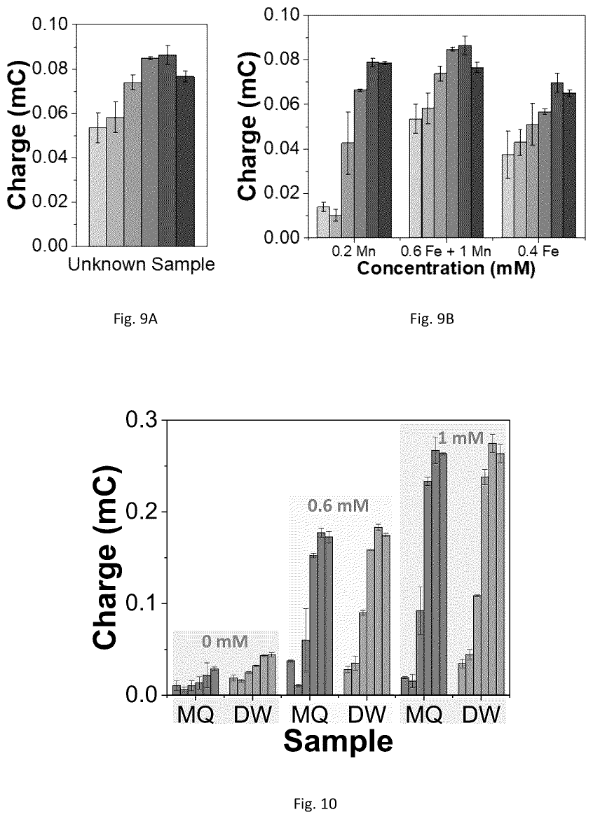

FIGS. 9A and 9B provide an example of how the pattern recognition method may be applied to determine the component of an unknown Mn²⁺ sample in presence of Fe²⁺. FIG. 9A is the pattern collected from an unknown sample possibly containing Mn²⁺ and Fe²⁺. FIG. 9B shows three selected patterns that are similar to the unknown sample. In both figures, the six bars correspond to electrode potentials of 0.9, 1.0, 1.1, 1.2, 1.3, and 1.4 V. In FIG. 9B, even though all three patterns have similar overall heights, the patterns have different shapes. It is difficult to use only one certain CA potential to accurately determine the concentration of Mn²⁺ since Fe²⁺ oxidation also transfers charge on the electrode at those potentials. But when looking at charges from multiple CA potentials as a pattern, the accuracy for determining the component is improved. In this example, the sample is clearly identified as a mixture of 0.6 mM Fe²⁺ and 1 mM Mn²⁺, because the unknown sample shares the same pattern. This example demonstrates the application of this method. More detailed patterns may be produced by adding more analysis channels (i.e., additional electrodes) to enhance the accuracy. In further embodiments, one or more additional feature in the water (e.g., pH, other metal ions) may be used to create an enhanced database for data comparison that would more closely reflect real-life scenarios.

### Multiplex Pattern Recognition Method of Mn²⁺ in a Real Drinking Water Sample

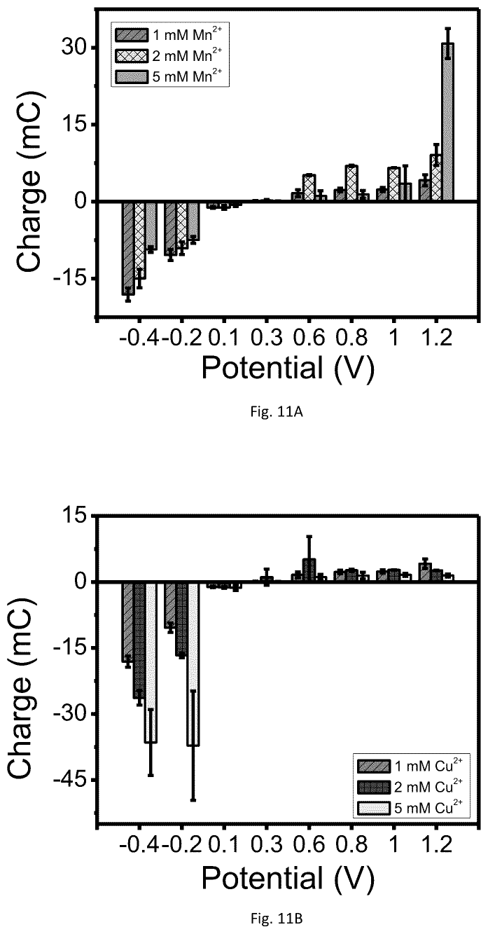

FIG. 10 shows the pattern recognition of Mn²⁺ in Milli-Q (MQ) water and a drinking water (DW) sample. The blank Milli-Q and drinking water samples without spiking additional Mn²⁺ give less similar patterns, which is due to the poor conductivity of Milli-Q water and the components originally existing in drinking water samples. After spiking 0.6 or 1.0 mM Mn²⁺ into the two matrices, the charges obtained from CA at the potential ranges from 1.1 to 1.4 V produced results that are easily comparable between Milli-Q water matrix and drinking water matrix, as shown in FIG. 10. When analyzing Mn²⁺ spiked samples using lower potentials at 0.9 and 1.0 V, the difference in charge between Milli-Q water matrix and drinking water matrix are significant at Mn²⁺ concentrations of 0.6 and 1.0 mM. These differences can be explained by the incomplete Mn²⁺ oxidation, as mentioned above. At 0.9 and 1.0 V, the potentials are not high enough to oxidize Mn²⁺, non-faradaic charges and other drinking water components contribute to the charge transfer during CA. Due to those reasons, the charges at 0.9 and 1.0 V are more susceptible to the interfering reagents in the drinking water matrix. Overall, it can be observed that when 0.6 and 1.0 mM Mn²⁺ are spiked into the two different sample matrices, the patterns between Milli-Q water matrix and drinking water matrix are highly similar. The recovery rates of Mn²⁺ in drinking water sample are shown in Table 2. Charges at the potentials of 1.2, 1.3 and 1.4 V were selected to calculate for recovery rates, due to the high correlation of the 3 calibration curves in Table 1. The recovery rates of both 0.6 and 1.0 mM Mn²⁺ samples were measured to be close to 100%. This indicates the utility of the multiplex pattern recognition method for Mn²⁺ detection.

TABLE 2
Recovery Rate of Drinking Water Samples Spiked with Mn²⁺.

Recovery Rate (%)

| CA Potential | 0.6 mM Mn²⁺ | 1.0 mM Mn²⁺ |
|---|---|---|
| 1.2 V | 105.6 | 100.9 |
| 1.3 V | 103.8 | 99.3 |
| 1.4 V | 102.4 | 99.8 |

### Example 2

A study was conducted to examine the ability of an embodiment to analyze sample solutions containing various concentrations of Mn²⁺, Fe²⁺, and Cu²⁺. The embodiment had eight gold working electrodes, a platinum counter electrode, and a salt bridge with reference electrode of Ag/AgCl in 3 M KCl. The setup and methods were otherwise similar to those described in Example 1.

Five sample solutions that provided three concentrations of Mn²⁺ and three concentrations of Cu²⁺ were analyzed:
1. 1 mM MnSO₄, 1 mM FeSO₄, 1 mM CuSO₄
2. 2 mM MnSO₄, 1 mM FeSO₄, 1 mM CuSO₄
3. 1 mM MnSO₄, 1 mM FeSO₄, 2 mM CuSO₄
4. 5 mM MnSO₄, 1 mM FeSO₄, 1 mM CuSO₄
5. 1 mM MnSO₄, 1 mM FeSO₄, 5 mM CuSO₄

The solutions were analyzed at electrode potentials of −0.4, −0.2, 0.1, 0.3, 0.6, 0.8, 1.0, and 1.2 V. FIG. 11A shows the multiplex electrode charge patterns for the three solutions with increasing concentrations of Mn²⁺, and FIG. 11B shows the charge patterns for the three solutions with increasing concentrations of Cu²⁺. It can be seen that unique charge patterns were obtained for the different solutions, demonstrating the ability of the embodiment to identify and quantify different metals in complex solutions.

All cited documents are incorporated herein by reference in their entirety.

### EQUIVALENTS

Those of ordinary skill in the art will recognize, or be able to ascertain through routine experimentation, equivalents to the embodiments described herein. Such equivalents are within the scope of the invention and are covered by the appended claims.

### REFERENCES

1. Chen, P., M. Culbreth, and M. Aschner, Exposure, epidemiology, and mechanism of the environmental toxicant manganese. Environ Sci Pollut Res Int, 2016. 23 (14): p. 13802-10.
2. Roels, H., et al., Epidemiological survey among workers exposed to manganese: effects on lung, central nervous system, and some biological indices. Am J Ind Med, 1987. 11 (3): p. 307-27.
3. Kim, H., et al., Exposing the role of metals in neurological disorders: a focus on manganese. Trends Mol Med, 2022. 28 (7): p. 555-568.
4. Health Canada, Guidelines for Canadian Drinking Water Quality. [Guidelines] 2019 [cited 2023 02/10].
5. WHO, Manganese in Drinking-water-Backgound document for development of WHO Guidelines for Drinking-water Quality. 2021, WHO: World Health Organization, Geneva, Switzerland.
6. Hutton, R. C., Application of inductively coupled plasma source mass spectrometry (ICP-MS) to the determination of trace metals in organics. Journal of Analytical Atomic Spectrometry, 1986. 1 (4): p. 259-263.
7. Locatelli, C. and G. Torsi, Voltammetric trace metal determinations by cathodic and anodic stripping voltammetry in environmental matrices in the presence of mutual interference. Journal of Electroanalytical Chemistry, 2001. 509 (1): p. 80-89.
8. Pei, Y., et al., A comparative study of electroanalytical methods for detecting manganese in drinking water distribution systems. Electrocatalysis, 2021. 12: p. 176-187.
9. Filipe, O. M. S. and C. M. A. Brett, Cathodic stripping voltammetry of trace Mn(II) at carbon film electrodes. Talanta, 2003. 61 (5): p. 643-650.
10. Kang, W. P., X.; Bange, A.; Haynes, E. N.; Heineman, W. R.; Papautsky, I., Determination of Manganese by Cathodic Stripping Voltammetry on a Microfabricated Platinum Thin-film Electrode. Electroanalysis, 2017. 29 (3): p. 686-695.
11. Jin, J.-Y., F. Xu, and T. Miwa, Cathodic Stripping Voltammetry for Determination of Trace Manganese with Graphite/Styrene-Acrylonitrile Copolymer Composite Electrodes. Electroanalysis, 2000. 12 (8): p. 610-615.
12. El-Desoky, H. S., I. M. Ismail, and M. M. Ghoneim, Stripping voltammetry method for determination of manganese as complex with oxine at the carbon paste electrode with and without modification with montmorillonite clay. Journal of Solid State Electrochemistry, 2013. 17 (12): p. 3153-3167.
13. Rusinek, C. A., et al., Bare and Polymer-Coated Indium Tin Oxide as Working Electrodes for Manganese Cathodic Stripping Voltammetry. Analytical Chemistry, 2016. 88 (8): p. 4221-4228.
14. Browne, K., et al., Ultrasensitive Electrochemical Phosphate Detection by Pyridine-zinc (II) Complex. Canadian Journal of Chemistry, 2023 (ja).

1. A method for analyzing water, comprising:
disposing a plurality of working electrodes and at least one counter electrode in the water;
simultaneously applying to each of the plurality of working electrodes a different potential selected from a range of potentials for a selected period of time;
obtaining chronoamperometry measurements of the plurality of working electrodes during the selected period of time;
using the chronoamperometry measurements to generate a pattern of electrical charge on the plurality of electrodes; and
correlating the pattern of electrical charge with one or more known patterns to perform one or more of: identify one or more analytes in the water; perform speciation of analytes; and determine one or more concentration of the one or more analytes in the water.

2. The method of claim 1, comprising using a salt bridge between the water and a reference electrode.

3. The method of claim 1, wherein the range of potentials is from about −1.5 V to about 1.5 V.

4. The method of claim 1, wherein the range of potentials is from about 0.9 V to about 1.4 V.

5. The method of claim 1, wherein increasing the selected period of time increases a detection sensitivity of analyzing the water.

6. The method of claim 1, wherein the selected period of time is up to about 1 minute.

7. The method of claim 1, wherein the selected period of time is up to about 5 minutes.

8. The method of claim 1, wherein the selected period of time is greater than 5 minutes.

9. The method of claim 1, wherein the analyte comprises one or more of manganese, copper, iron, lead, arsenic, barium, chromium, nickel, radium, uranium, and vanadium.

10. The method of claim 1, comprising using two or more selected ranges of potentials;
simultaneously applying to each of the plurality of working electrodes a different potential selected from the two or more selected ranges of potentials for a selected period of time;
obtaining chronoamperometry measurements of the plurality of working electrodes during the selected period of time;
using the chronoamperometry measurements to generate a pattern of electrical charge on the plurality of electrodes corresponding to each of the two or more selected ranges of potentials; and
correlating the patterns of electrical charge with known patterns to perform one or more of: identify two or more analytes in the water; perform speciation of analytes; and determine concentration of the two or more analytes in the water.

11. The method of claim 1, wherein the water is drinking water, municipal water, well water, river water, lake water, or spring water.

12. The method of claim 1, wherein the water is a water sample.

13. Apparatus for analyzing water, comprising:
a plurality of working electrodes and at least one counter electrode adapted to be in contact with the water;
a source of electrical potential difference that simultaneously applies to each of the plurality of working electrodes a different potential selected from at least one range of potentials;
a multichannel device that obtains chronoamperometry measurements from the plurality of working electrodes;
a processor that uses the chronoamperometry measurements to generate a pattern of electrical charge on the plurality of electrodes and correlates the pattern of electrical charge with one or more known patterns to perform one or more of: identify one or more analytes in the water, perform speciation of analytes, and determine one or more concentration of the one or more analytes in the water.

14. The apparatus of claim 13, comprising a salt bridge between the water and a reference electrode.

15. The apparatus of claim 13, wherein the range of potentials is from about −1.5 V to about 1.5 V.

16. The apparatus of claim 13, wherein the range of potentials is from about 0.9 V to about 1.4 V.

17. The apparatus of claim 1, wherein the multichannel device obtains the chronoamperometry measurements from the plurality of working electrodes for a selected period of time;
wherein increasing the selected period of time increases a detection sensitivity of analyzing the water.

18. The apparatus of claim 13, wherein the analyte comprises one or more of manganese, copper, iron, lead, arsenic, barium, chromium, nickel, radium, uranium, and vanadium.

19. The apparatus of claim 13, wherein the water is drinking water, municipal water, well water, river water, lake water, or spring water.

20. The apparatus of claim 13, implemented for in-line analysis of the water.
